(19) United States
(12) Patent Application Publication
(10) Pub. No.: US 2007/0048786 A1
(43) Pub. Date: Mar. 1, 2007

(54) Systems and methods for fractionation of protein mixtures

(76) Inventors: Arnon Chait, Bay Village, OH (US); Boris Y. Zaslavsky, Solon, OH (US)

Correspondence Address: PEARNE & GORDON LLP, 1801 EAST 9TH STREET, SUITE 1200, CLEVELAND, OH, 44114-3108, US

(21) Appl. No.: 11/217,166

(22) Filed: Sep. 1, 2005

(51) Int. Cl. G01N 33/53; C07K 1/10

(52) U.S. Cl. 435/7.1; 530/412

(57) ABSTRACT

Methods for proteomics analysis, including fractionation or separation of one or more biomolecules that exist in a mixture, for example, using aqueous multi-phase partitioning, which results in isolation of one or more preselected biomolecules, followed by further proteomics analysis.

### FIELD OF THE INVENTION

The present invention is generally related to the separation, fractionation, and/or segregation of one or more biomolecules that exist in a mixture, typically performed in conjunction with general methods for discovering biomarkers related to a physiological condition referred to as proteomics techniques. More particularly, the invention is related to developing methods for fractionation using, for example, aqueous multi-phase partitioning, which result in isolation of select biomolecule or biomolecules. More particularly, these methods also provide fractionation according to physico-chemical parameters that are different than those typically used in proteomics techniques, thereby providing additional means to simplify the mixture of biomolecules prior to analysis. Moreover, the isolation of select biomolecules or biomolecules can be conducted separately or in a single step using methods and techniques of the present invention.

### BACKGROUND OF THE INVENTION

This invention relates generally to the fractionation of biomolecules, complexes comprising biomolecules or analogous species thereof which is typically performed before other analyses collectively referred to as proteomics techniques. Proteomics techniques are commonly used to identify and/or isolate a subset of biomolecules with expression levels and/or with structural and/or functional properties specific for the purpose of analysis, in particular, specific for protein markers of a disease and/or physiological state of a living organism under investigation.

Many diseases and/or pathological processes are caused by disfunction or disregulation of certain proteins. These disease-related proteins may have the structures altered relative to their “normal” counterparts and/or may be expressed in larger (upregulated expression) or lower (downregulated expression) quantities in a given disease state than under “normal” physiological conditions. Proteins with altered structure and/or function may serve as protein markers associated with a particular human or animal disease, either as diagnostics for earlier detection of diseases, as monitors of disease progression and/or treatment, or as drug or antibody targets for treatment.

In many cases the particular proteins of relevance to a given pathological process are unknown. Identification of such proteins would be useful for development of new diagnostic tests and/or new drugs.

For a biomarker or set of biomarkers to be of clinical value it must be derived from a readily obtainable sample. Although urine is widely used in diagnostics, blood serum or plasma is potentially the most valuable source for biomarkers [Anderson, N. L., Anderson, N. G., Mol. Cell. Proteomics, 2002, 1, 845-867]. Since serum constantly perfuses tissues, it might be expected that the onset or presence of disease may be determined by measuring the altered presence or abundance of the constituent protein species in serum. For example, increased serum levels of prostate-specific antigen [Grossklaus, D. J., Smith, J. A., Shappel, S. B., Coffey, C. S., Chang, S. S., Cookson, M. S., Urol. Oncol., 2002, 7, 195-198] and CA125 [Whitehouse, C., Solomon, E., Gynecol. Oncol., 2003, 88m S152-157] are routinely used for detection of cancer in the prostate and ovary, respectively.

Serum (or plasma) has a high protein content with many of the proteins being secreted and shed from cells and tissues [Sasaki, K., Sato, K., Akiyama, Y., Yanagihara, K., Oka, M., Yamaguchi, K., Cancer Res., 2002, 62, 4894-4898; Kennedy, S., Biomarkers, 2002, 7, 269-290; Adkins, J. N., Varnum, S. M., Auberry, K. J., Moore, R. J., Angell, N. H., Smith, R. D., Springer, D. L., Pounds, J. G., Mol. Cell. Proteomics, 2002, 1, 947-955], however, the protein content of serum is dominated by a handful of proteins such as albumin, transferrin, haptoglobin, immunoglobulins, and lipoproteins [Anderson, N. L., Anderson, N. G., Mol. Cell. Proteomics, 2002, 1, 845-867]. These proteins constitute about 90-94% of all the total amount of serum proteins. The concentration range of serum proteins is likely to span more than 10 orders of magnitude, which separates albumin from the rarest proteins currently measured clinically [Anderson, N. L., Anderson, N. G., Mol. Cell. Proteomics, 2002, 1, 845-867]. This large dynamic range exceeds the analytical capabilities of traditional proteomic methods making the detection of lower-abundance serum proteins extremely challenging. The reduction of sample complexity by depletion or decrease of the level of abundant proteins is thus an essential first step in the analysis of serum proteome [Righetti, P. G., Castagna, A., Antonioli, P., Boschetti, E., Electrophoresis, 2005, 26, 297-319].

Affinity methods, such as anti-human serum albumin antibody columns, protein A/G, have been developed to remove highly abundant proteins, such as albumin and immunoglobulins from serum prior to two-dimensional gel electrophoresis (2-DE) or two-dimensional high-performance liquid chromatography (2D-HPLC) or liquid chromatography (LC) coupled with mass spectrometric analysis [Bjorhall, K., Miliotis, T., Davidsson, P., Proteomics, 2005, 5, 307-317].

One of the fundamental oversights of currently existing serum depletion methodologies is that many important low-molecular-weight proteins or peptides may be concomitantly removed by this sample preparation process [Zhou, M., Lucas, D. A., Chan, K. C., Issaq, H. J., Petricoin, E. F., Liotta, L. A., Veenstra, T. D., Conrads, T. P., Electrophoresis, 2004. 25, 1289-1298; Harper, R. G., Workman, S. R., Schuetzner, S., Timperman, A. T., Sutton, J. N., Electrophoresis, 2004, 25, 1299-1306; Petricoin, E. F., Ornstein, D. K., Liotta, L. A., Urol. Oncol., 2004, 22, 322-328]. Indeed it is in general highly beneficial for any fractionation method to retain as much as possible of the protein content of original sample even if certain high abundance proteins are selectively removed.

Yet another simplification of the sample before analysis could be derived from fractionation of the mixture of proteins into two or more subsets according to a physic-chemical parameter or parameters that are different than those used in subsequent proteomics analysis techniques. For example, common proteomics techniques fractionate the samples using two-dimensional SDS gel electrophoresis, in which the proteins are separated by molecular weight and their net electrical charge. Thus a fractionation method that is different or orthogonal to separation according to molecular weight and charge could be useful for further simplification of the sample.

Yet another consideration in selecting fractionation methods is their ability to segregate proteins according to their state in the sample as individual proteins or as proteins that are bound to other proteins or other ligands or drugs. Loss of an ability to form complexes with other proteins (e.g., in a biological signaling cascade), differentially between positive and control samples of a particular physiological condition, could potentially identify a protein as a biomarker candidate. Such information could also be useful for discovery of protein markers which are connected with drug actions, including toxicology applications and alike.

It is the objective of the present invention to provide a new method for fractionation of proteins and other related compounds, of substantial utility prior to further multi-dimensional separation and identification of protein markers, drug targets, and alike. The purpose of the fractionation is to reduce the total number of proteins in the experimental sample obtained from any biological fluid or cellular matter, and/or to enrich the proteins of interest for the purpose of analysis, while maintaining the natural protein-protein and protein-ligand complexes, and preserving integrity of all the proteins and their complexes in solution ready for further analysis. This and other objectives of the invention will become apparent in the detailed description below.

### SUMMARY OF THE INVENTION

A method for proteomics analysis, including fractionation of a mixture of biomolecules, said mixture containing at least a first biomolecule and a second biomolecule, said method comprising the steps of: providing a multi-phase partitioning system, combining a sample containing said mixture of biomolecules with said system, causing or permitting said system to separate into at least a first phase and a second phase, wherein said first biomolecule is preferentially segregated into said first phase, selecting said first phase or said second phase, and performing further proteomics analysis on said selected phase.

The present invention provides a technique for fractionation of a mixture of biomolecules, including those interacting with other biomolecules that are originated from an experimental sample that is obtained from any biological fluid or cell matter. Fractionation of the mixtures may be performed by single-step or multiple-step extraction, or liquid-liquid partition chromatography for segregating of a given protein or a subset of proteins, and/or their complexes with other compounds enriched in the proteins of interest for the purpose of analysis for further isolation, purification, and identification.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For the purposes of clarity, not every component is labeled in every figure, nor is every component of each embodiment of the invention shown where illustration is not necessary to allow those of ordinary skill in the art to understand the invention. In the figures:

FIG. 1. Electrophoregram of the protein mixtures in the two phases fractionated according to Example 1.

FIG. 2. SDS-PAGE two-dimensional gel electrophoresis images of the protein mixtures in the two phases fractionated according to Example 1.

FIG. 3. Zoomed sections corresponding to the albumin rich region in the gel images of FIG. 2.

FIG. 4. Electrophoregram of the protein mixtures in the two phases fractionated according to Example 2.

FIG. 5. Electrophoregram of the protein mixtures in the two phases fractionated according to Example 3.

FIG. 6. SDS-PAGE two-dimensional gel electrophoresis image of the protein mixtures in the two phases fractionated according to Example 3.

FIG. 7. Zoomed sections corresponding to the IgG heavy chain rich region in the gel images of FIG. 4.

FIG. 8. SDS-PAGE two-dimensional gel electrophoresis images of the protein mixtures in the two phases fractionated according to Example 4.

FIG. 9. Zoomed sections of the same regions in gel images of the two phases FIG. 8.

FIG. 10. Electrophoregram of the rabbit protein mixture in the top phase of the second system fractionated according to Example 5.

FIG. 11. Electrophoregram of the bovine protein mixture in the top phase of the second system fractionated according to Example 5.

FIG. 12. Electrophoregram of the rat protein mixture in the top phase of the second system fractionated according to Example 5.

### DETAILED DESCRIPTIONS OF THE PREFERRED EMBODIMENTS OF THE INVENTION

Selected Definitions

As used in the specification and the appended claims, the singular forms “a,” “an” and “the” include plural referents unless the context clearly dictates otherwise. Thus, for example, reference to “a biomolecule” can include mixtures of a biomolecule, and the like.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

As used herein, “or” is understood to mean inclusively or, i.e., the inclusion of at least one, but including more than one, of a number or list of elements. Only terms clearly indicated to the contrary, such as “exclusively or” or “exactly one of,” will refer to the inclusion of exactly one element of a number or list of elements.

“Optional” or “optionally” means that the subsequently described event or circumstance may or may not occur, and that the description includes instances where said event or circumstance occurs and instances where it does not.

“Proteome” refers to the total or subset of the protein content in a biological sample. “Proteomics analysis” refers to analysis methodologies that attempt to identify changes in protein amount or structure that correspond to differences in the physiological state of the sample. These analysis methodologies typically involve one or more fractionation steps of the sample to simplify the content of the proteome, followed by isolation and identification of the proteins that are deemed different between the samples.

“Analyte,” “analyte molecule,” or “analyte species” refers to a molecule, typically a macromolecule, such as a polynucleotide or polypeptide, whose presence, amount, and/or identity are to be determined.

“Antibody,” as used herein, means a polyclonal or monoclonal antibody. Further, the term “antibody” means intact immunoglobulin molecules, chimeric immunoglobulin molecules, or Fab or F(ab′)₂ fragments. Such antibodies and antibody fragments can be produced by techniques well known in the art, which include, for example, those described in Harlow and Lane (Antibodies: A Laboratory Manual, Cold Spring Harbor Laboratory, Cold Spring Harbor, N.Y. (1989)), Kohler et al. (Nature 256: 495-97 (1975)), and U.S. Pat. Nos. 5,545,806, 5,569,825 and 5,625,126, each incorporated herein by reference. Correspondingly, antibodies, as defined herein, also include single chain antibodies (ScFv), which may comprise linked V_{H} and V_{L} domains and which may retain the conformation and the specific binding activity of the native idiotype of the antibody. Such single chain antibodies are well known in the art and can be produced by standard methods. See, e.g., Alvarez et al., Hum. Gene Ther. 8: 229-242 (1997)). The antibodies of the present invention can be of any isotype, for example, IgG, IgA, IgD, IgE and IgM.

“Aqueous,” as used herein, refers to the characteristic properties of a solvent/solute system wherein the solvating substance has a predominantly hydrophilic character. Examples of aqueous solvent/solute systems include those where water, or compositions containing water, are the predominant solvent.

“Partitioning system”, as used herein, refers to any material having at least two phases, sections, areas, components, or the like, at least two of which can interact differently with at least one species to which they are exposed. For example, a partitioning system can include a multi-phase system such as a multi-phase liquid system, e.g., an aqueous/non-aqueous system or an aqueous multi-phase system (defined below) to which one or more species can be exposed and optionally dissolved, at least some of which species can interact differently with different phases. For example, a particular species may have a greater affinity for one phase rather than another phase to the extent that a multi-phase partitioning system can isolate a species from a mixture, or cause a species to partition at least in some way differently between the phases.

“Aqueous multi-phase system,” as used herein, refers to an aqueous system which consists of greater than one aqueous phase in which an analyte species can reside, and which can be used to characterize the structural state of the analyte species according to the methods described herein. For example, an aqueous multi-phase system can separate at equilibrium into two, three, or more immiscible phases. Aqueous multi-phase systems are known in the art and this phrase, as used herein, is not meant to be inconsistent with accepted meaning in the art. Examples of various aqueous multi-phase systems, and their compositions, are described more fully below.

An “interacting component” means a component, such as a phase of multi-phase system, that can interact with a species and provide information about that species (for example, an affinity for the species). Multiple interacting components, exposed to a species, can define a system that can provide a “relative measure of interaction” between each component and the species. An interacting component can be aqueous or non-aqueous, can be polymeric, organic (e.g. a protein, small molecule, etc.), inorganic (e.g. a salt), or the like, or any combination thereof. A set of interacting components can form a system useful in and in part defining any experimental method which is used to characterize the structural state of a species such as an analyte species according to the methods described herein. Typically, a system of interacting components can measure the relative interaction between the species and at least two interacting components. An aqueous multi-phase system is an example of a system of interacting components, and it is to be understood that where “aqueous system” or “aqueous multi-phase system” is used herein, this is by way of example only, and any suitable system of interacting components can be used.

Where aqueous two-phase and aqueous multi-phase systems are described herein, it is to be understood that other systems, as used herein, systems analogous to those comprising only aqueous solutions or suspensions can be used. In this aspect, multi-phase systems also refers to related techniques that rely on differential affinity of the biomolecule to one media versus another, wherein the transport of the biomolecule between one medium and, optionally, another medium occurs in an aqueous environment. Examples of such multi-phase systems include, but are not limited to systems for liquid-liquid partition chromatography, as are known to those of ordinary skill in the art.

“Relative measure of interaction”, with reference to a particular species as used herein means the degree to which the species interacts with another species or with a phase of a multi-phase system in a relative sense. For example, a particular species may have a greater affinity for one phase of a multi-phase system rather than another phase or phases, the degree to which it interacts with or resides in, that phase as opposed to other phases defines its relative measure of interaction. Relative measures of interaction, in the context of the present invention, are generally determined in a ratio metric manner, rather than an absolute manner. That is, where a species can interact with each phase of a two-phase system but resides more preferably in one than the other, the present invention typically makes use of information as to the ratio of concentration of the species in each of the two phases, but not necessarily of the absolute concentration of the species in either phase.

“Partition coefficient,” as used herein, refers to the coefficient which is defined by the ratio of chemical activity or the concentrations of a species in two or more phases of a multi-phase system at equilibrium. For example, the partition coefficient (K) of an analyte in a two-phase system is defined as the ratio of the concentration of analyte in the first phase to that in the second phase. For multi-phase systems, there are multiple partition coefficients, where each partition coefficient defines the ratio of species in first selected phase and a second selected phase. It will be recognized that the total number of partition coefficients in any multi-phase system will be equal to the total number of phases minus one.

“Covalent” bond or interaction refers to a chemical bond formed by sharing of one or more electrons.

“Bind,” as used herein, means the well understood receptor/ligand binding, as well as other nonrandom association between an a biomolecule and its binding partner.

“Specifically bind,” as used herein, describes a binding partner or other ligand that does not cross react substantially with any biomolecule other than the biomolecule or biomolecules specified. Generally, molecules which preferentially bind to each other are referred to as a “specific binding pair.” Such pairs include, but are not limited to, an antibody and its antigen, a lectin and a carbohydrate which it binds, an enzyme and its substrate, and a hormone and its cellular receptor. As generally used, the terms “receptor” and “ligand” are used to identify a pair of binding molecules. Usually, the term “receptor” is assigned to a member of a specific binding pair, which is of a class of molecules known for its binding activity, e.g., antibodies. The term “receptor” is also preferentially conferred on the member of a pair that is larger in size, e.g., on lectin in the case of the lectin-carbohydrate pair. However, it will be recognized by those of skill in the art that the identification of receptor and ligand is somewhat arbitrary, and the term “ligand” may be used to refer to a molecule which others would call a “receptor.” The term “anti-ligand” is sometimes used in place of “receptor.”

“Molecule—molecule interaction”, such as biomolecule—biomolecule interaction, protein—protein interaction, and the like means an interaction that typically is weaker than “binding”, i.e., an interaction based upon hydrogen bonding, van der Walls binding, London forces, and other non-covalent interactions that contribute to an affinity of one molecule for another molecule, which affinity can be assisted by structural features such as the ability of one molecule to conform to another molecule or a section of another molecule. Molecule—molecule interactions can involve binding, but need not.

“Biomolecule,” as used herein, means a molecule typically derived from an organism, and which typically includes building blocks including nucleotides, and the like. Examples include peptides, polypeptides, proteins, protein complexes, nucleotides, oligonucleotides, polynucleotides, nucleic acid complexes, saccharides, oligosaccharides, carbohydrates, lipids, fatty acids, sugars, as well as combinations, enantiomers, metabolites, complexes, homologs, analogs, derivatives and/or mimetics thereof. In the present invention the word “protein” is used to define a protein or any biomolecule type defined herein.

“Species”, as used herein, refers to a molecule or collection of molecules. For example, an inorganic chemical, an organic chemical, a biomolecule, or the like. In the present invention, species generally are biomolecules.

“Structure,” “structural state,”, “configuration” or “conformation,” as used herein, all refer to the commonly understood meanings of the respective terms, for example, as they apply to biomolecules such as proteins and nucleic acids, as well as pharmacologically active small molecules. In different contexts, the meaning of these terms will vary, as is appreciated by those of skill in the art. The structure or structural state of a molecule refers generally not to the building blocks that define the molecule but the spatial arrangement of these building blocks. The configuration or conformation typically defines this arrangement. For instance, the use of the terms primary, secondary, tertiary or quaternary, in reference to protein structure, have accepted meanings within the art, which differ in some respects from their meaning when used in reference to nucleic acid structure (see, e.g., Cantor and Schimmel, Biophysical Chemistry, Parts I-III). Unless otherwise specified, the meanings of these terms will be those generally accepted by those of skill in the art.

“Physiological conditions”, as used herein, means the physical, chemical, or biophysical state of an organism. As most typically used in the context of the present invention, physiological condition refers to a normal (e.g., healthy in the context of a human) or abnormal (e.g., in a diseased state in the context of a human) condition.

“Marker” as used herein, is a biomolecule, whose differential expression level or differential structure corresponding to different physiological conditions, makes it a potential carrier of information regarding a physiological state of a biological environment within which it resides. A marker can exhibit at least two different properties or values of a specific property or properties (e.g., expression level, structural conformation, binding affinity for another species, etc.) that correspond to and that represent information regarding the two or more physiological states of environments within which they reside.

### EMBODIMENTS

The present invention involves techniques for fractionation of a multi-species mixture originated from a biological material, such as biological fluid, tissue, or cells. More particularly, in one embodiment, the invention is related to depletion or segregation of highly abundant proteins from a mixture while maintaining these proteins in a form available for further fractionation and analysis, and to fractionation of a mixture into two or more fractions of different protein composition for further fractionation and analysis. In another embodiment, this fractionation may also serve to provide an additional dimension for proteome fractionation that is based on different physico-chemical basis other than solely on the size and charge of each of the proteins in the mixture. In yet another embodiment, the methods of the present invention can provide means to simultaneously deplete or segregate highly abundant proteins from a mixture, while fractionating the entire mixture content according to differences in the physic-chemical properties of the partitioning system and the structural or other properties of the proteins comprising the mixture. In another embodiment, aliquots from phases of such partitioning systems could be introduced into systems with same or different properties to focus, change, enhance, or amplify the observed partitioning behavior of the mixture.

Methods of the present invention can be useful for sample preparations for further fractionation and/or analysis for detecting, classifying, and/or predicting changes in the composition of the mixture of biomolecules or molecules that interact with biomolecules associated with a particular disease or physiological state of a living organism, cells, tissues, or biological liquids. It is to be understood that this invention is not limited to specific methods, specific solutions, or to particular devices, as such may, of course, vary. It is also to be understood that the terminology used herein is for the purpose of describing particular embodiments only and is not intended to be limiting.

Proteomics analysis is commonly comprised of the following steps:

1. Sanple preparation. This step is comprised of obtaining a biological sample and performing steps specific to each type of sample in preparation for subsequent analysis. Such preparation steps may include cell membrane disruption and solubilization, removal of certain whole blood components such as platelets, etc.

2. Sample fractionation. Fractionation refers to further simplification of the protein mixture of the sample such that differences in the proteome of samples corresponding to different physiological origins could be revealed and identified in subsequent steps. Fractionation typically includes separation of the mixture of proteins according to one or more physico-chemical properties, such as net electrical charge or molecular weight. Using gel electrophoresis provides for means to separate the mixture according to molecular weight in one dimension, while separation according to charge could be accomplished using an established pH gradient that provides for migration of proteins to their isoelectric points. In one common technique referred to as two-dimensional gel, a mixture is separated according to the above two dimensions and stained to reveal individual proteins. The presence of dark stained spots at a specific location in charge/molecular weight could correspond to individual proteins, but the same spot may be occupied by more than one protein, making differential analysis difficult.

Fractionation is sample specific. For example, serum or plasma samples contain several high abundance proteins, such as albumin, haptoglobin, immunoglobulins, and lipoproteins. The presence of such proteins in large quantities masks other less abundant proteins which possess similar properties such as molecular weight or net electrical charge when a two-dimensional gel analysis is performed. Thus, the purpose of early fractionation steps may include depletion or removal of such proteins from the mixture so that other less abundant proteins may be revealed in subsequent fractionation steps. For example, depletion of at least 90% or more of abundant proteins in serum provides advantages for further fractionation.

Fractionation according to more than the standard charge/molecular weight dimensions is also highly desirable. For example, serum may contain 500,000 proteins or more, and a simple two-dimensional fractionation may not be sufficient to simplify the mixture enough to detect differences between individual proteins. Indeed, the so-called “orthogonal fractionation” typically refers to factionation of the mixture according to different principles than size or charge alone. For example, fractionation that is based on the protein overall hydrophobicity level, on affinity to certain ligands, on ability to participate in protein-protein interactions and the like are potentially highly useful and can be used alone or in conjunction with size/charge based fractionation to further simply the mixture of proteins.

Finally, fractionation techniques that can be flexibly used in different sequences are also of interest. For example, a first step of fractionation may involve depletion of albumin from a serum mixture, which is then followed by a second step in which immunoglobulins are removed, or by a second step which fractionate the sample according to hydrophobicity, the results of which are then fractionated by size/charge.

3. Sample analysis. In this step techniques are used to identify differences between the proteome samples corresponding to different physiological conditions. For example, dark gel spots that appear differently at the same size/charge locations can be physically cut and analyzed. Analysis typically involves mass spectrometry and similar techniques to identify the protein(s).

The multi-phase partitioning system of the invention yields at least a first phase and a second phase. One of said phases is then selected and further proteomics analysis is then performed on said selected phase; the proteomics analysis can be selected from the group consisting of fractionation using a multi-phase partitioning system, gel electrophoresis, chromatography, adsorption chromatography, partition chromatography, high pressure liquid chromatography (HPLC), paper chromatography, affinity separation utilizing ligands fixed on a substrate, other similar fractionation techniques, techniques to identify specific proteins or protein groups, the techniques including mass spectrometry, the use of protein chips, and techniques similar to these two techniques.

In one embodiment, the present invention is a fractionation technique that can be used to deplete one or more abundant or other desired proteins from a mixture. In another embodiment, the present invention is a fractionation technique for depleting one or more abundant proteins from a mixture that operate with similar efficiency on samples obtained across different species. In another embodiment, the present technique provides a flexible fractionation dimension that is not solely dependent on charge or molecular weight. In yet another embodiment, the present invention enable sequential fractionation by performing a given fractionation according to techniques described herein, followed by removal of a desired aliquot after such fractionation and introducing it into a second fractionation. The sequence can then be repeated as desired to enable further focusing, depletion, etc. In yet another embodiment, the present invention provides an aqueous fractionation environment which preserves protein structure or at least does not denature proteins. Thus, fractionation dimensions that may reflect addition or loss of certain protein-ligand or protein-protein complexes can be available through the use of techniques described by the present invention. In yet another embodiment, the technique of the present invention preserves the entire proteome during fractionation for further analysis. For example, while albumin can be depleted from the proteome in one embodiment, it can be harvested for a separate analysis, e.g., for a study of proteins that might be carried by albumin and are of potential importance to a proteomics analysis.

Aqueous two-phase systems arise in aqueous mixtures of different water-soluble polymers (as known in the art) or a single polymer (as known in the art) and a specific salt (the salt can be any salt as known in the art, preferably an inorganic salt, such as NaCl or CaCl, or a salt wherein the positive ion is one of Na, K, Li, Ca, Mg, Ba, Zi, Al, Mn, etc and the negative ion is one of F, Cl, Br, I, S, etc, or wherein the ion is citrate, sulfate, nitrate, phosphate, carbonate, borate, ammonium, etc). When two certain polymers, e.g., dextran (Dex) and polyethylene glycol (PEG), or a single certain polymer and a certain inorganic salt, e.g. polyvinylpyrrolidone (PVP) and sodium sulfate, are mixed in water above certain concentrations, the mixture separates into two immiscible aqueous phases. There is a discrete interfacial boundary separating two phases, one rich in one polymer and the other rich in the other polymer or inorganic salt. The aqueous solvent in both phases provides media suitable for biological products. More particularly, the abundance of an aqueous solvent is of specific value for maintaining non-denaturing conditions for the proteins during fractionation, thus potentially enabling fractionation based on protein-protein interactions or their loss and other structure-sensitive biological processes that may be of interest. Two-phase systems can be generalized to multiple phase system by using different chemical components, and aqueous systems with a dozen or more phases have been mentioned in the literature.

When a solute is introduced into such a two-phase system, it distributes between the two phases. Partitioning of a solute is characterized by the partition coefficient K defined as the ratio between the concentrations of the solute in the two imiscible phases at equilibrium. It was previously shown that phase separation in aqueous polymer systems results from different effects of two polymers (or a single polymer and a salt) on the water structure (B. Zaslavsky, Aqueous Two-Phase Partitioning: Physical Chemistry and Bioanalytical Applications, Marcel Dekker, New York, 1995). As the result of the different effects on water structure, the solvent features of aqueous media in the coexisting phases differ from one another. The difference between phases can be demonstrated by dielectric, solvatochromic, potentiometric, and partition measurements.

The basic rules of solute partitioning in aqueous two-phase systems were shown to be similar to those in water-organic solvent systems (Zaslavsky). However, what differences do exist in the properties of the two phases in aqueous polymer systems are very small relative to those observed in water-organic solvent systems, as should be expected for a pair of solvents of the same (aqueous) nature. Importantly, the small differences between the solvent features of the phases in aqueous two-phase or multi-phase systems can be modified so as to amplify the observed partitioning that results when certain structural features are present.

It is known that the polymer and salt compositions of each of the phases depend upon the total polymer and salt composition of an aqueous two-phase system. The polymer and salt composition of a given phase, in turn, governs the solvent features of an aqueous media in this phase. These features include, but are not limited to, dielectric properties, solvent polarity, ability of the solvent to participate in hydrophobic hydration interactions with a solute, ability of the solvent to participate in electrostatic interactions with a solute, and hydrogen bond acidity and basicity of the solvent. All these and other solvent features of aqueous media in the coexisting phases may be manipulated by selection of polymer and salt composition of an aqueous two-phase system. These solvent features of the media govern the sensitivity of a given aqueous two-phase system toward a particular type of solvent accessible chemical groups in the receptor. This sensitivity, type, and topography of the solvent accessible groups in two different proteins, for example, determine the possibility of separating proteins in a given aqueous two-phase system.

Currently the field lacks the theory capable of relating the polymer and salt composition of a system to the sensitivity of the aqueous media in the two phases toward different solvent accessible chemical groups in the biomolecules. This sensitivity is of paramount importance when, for example, subtle differences are being detected between the conformational changes in a receptor induced by binding of closely related chemical compounds. However, by utilizing a wide variety of different trial or experimental conditions to screen each protein mixture, conditions displaying differences between the composition of the mixtures and differences in the structures of the constituents of the mixtures can be obtained reliably without the need to fully understand the underlying theory of aqueous two-phase partitioning, or any of the other related or substitutable techniques.

Selection and modification of the types, as reflected in, for example, the chemical nature, structure, and molecular weight, of the phase-forming polymers and the concentration of the polymers can be used to vary the properties of the phases. In addition, the composition of the phases can also be changed by the addition of inorganic salts and/or organic additives. Changes to the composition of the phases can alter the properties of the phases. Examples of types of aqueous two-phase systems that are useful for separation of the mixtures of biomolecules include, but are not limited to, dextran/PEG, dextran/polyvinylpyrrolidone, PEG/salt, and polyvinylpyrrolidone/salt.

Biomolecules such as proteins, nucleic acids or other also distribute between the two phases when placed into such a system. This partitioning of a biomolecule between the two phases is fairly simple. In some respects, it is similar to extraction as is normally in the chemical arts. For example, in the case where phase-forming polymers are used, solutions comprising one or more of the two polymers and the biomolecule are mixed together such that both phase-forming polymers and the biomolecule are mixed. The resulting solution is resolved and the two-phase system is formed. Optionally, centrifugation can be used to enhance separation of the phases. Optionally, the formation of a two-phase system and the partitioning of solutes in such a system could be accomplished in a continuous manner using chromatographical techniques on a large scale, e.g., using liquid-liquid partition chromatography, or on a microscale, e.g., using a continuous microfluidic device. In the latter case, mixing could be accomplished using diffusion alone and active mixing and centrifugation are not necessary. It will be recognized by those of skill in the art that partitioning behavior of a biomolecule may be influenced by many variables, such as the pH, the polymers used, the salts used, other factors relating to the composition of the system, as well as other factors such as temperature, volume, etc. Optimization of these factors for desired effects can be accomplished by routine practice by those of skill in the relevant arts in combination with the current disclosure.

Evaluation of data from partitioning of a mixture of biomolecules can involve use of the partition coefficient (“K”), which is defined as the ratio between the concentrations of the biomolecule in the two immiscible phases at equilibrium. For example, the partition coefficient, K, of a protein is defined as the ratio of the protein in first phase to that in the second phase in a biphasic system. When multiple phase systems are formed, there can be multiple independent partition coefficients that could be defined between any two phases. From mass balance considerations, the number of independent partition coefficients will be one less than the number of phases in the system.

It will be recognized that the partition coefficient K for a given biomolecule of a given conformation will be a constant if the conditions and the composition of the two-phase system to which it is subjected remain constant. Thus, if there are changes in the observed partition coefficient K for the protein upon addition of a potential binding partner, these changes can be presumed to result from changes in the protein structure caused by formation of a protein-binding partner complex. In another case changes to the K value could indicate structural changes to the protein, e.g., changes in the conformation or the type and topography of the solvent-exposed residues due to e.g., phosphorylation, oxidation, deamidation, single residue mutations, etc. “K”, as used herein, is used as specifically mathematically defined below, and in all instances also includes, by definition, any coefficient representing the relative measure of interaction between a species and at least two interacting components.

In order to determine the partition coefficient K of a protein or a mixture of a protein with another compound, or a mixture of different proteins and compounds with which these proteins may interact to be analyzed, concentrated stock solutions of all the components (polymer 1, e.g., dextran; polymer 2, e.g., PEG, polyvinylpyrrolidone, salts, etc.) in water can be prepared separately. The stock solutions of phase polymers, salts, and the protein mixture can be mixed in the amounts and conditions (e.g., pH from about 3.0 to about 9.0, temperature from about 4° C. to 60° C., salt concentration from 0.001 to 5 mole/kg) appropriate to bring the system to the desired composition and vigorously shaken. The system can then be allowed to equilibrate (resolve the phases). Equilibration can be accomplished by allowing the solution to remain undisturbed, or it can be accelerated by centrifugation, e.g., for 2-30 minutes at about 1000 to 4000 g or higher. Aliquots of each settled (resolved) phase can be withdrawn from both the upper and lower phases. The concentration of biomolecule can be determined for both the upper and lower phases. The batch-like process illustrated above could be substituted by other processes, e.g., liquid-liquid partitioning chromatography or a microfluidics device performing the same, as known to those skilled in the art.

Different assay methods may be used to determine the concentration of the biomolecules in each phase. The assays will depend upon the identity and type of biomolecules present. Examples of suitable assay techniques include, but are not limited to, spectroscopic, immunochemical, chemical, fluorescent, radiological and enzymatic assays. When the biomolecule is a peptide or protein, the common peptide or protein detection techniques can be used. These include direct spectrophotometry (monitoring the absorbance at 280 nanometers) and dye binding reactions with Coomassie Blue G-250 or fluorescamine, o-phthaldialdehyde, or other dyes and/or reagents. Alternatively, if the protein is either an antibody or an antigen, immunochemical assays can also be used.

When a mixture of different proteins and other compounds is examined by partitioning the total concentration of the proteins assayed in each phase will depend upon the particular assay being used, since contribution of each protein in the analytical signal produced by a given assay may vary depending upon the particular protein and particular assay.

Protein mixtures from an experimental sample and from the reference sample may be subjected to partition in a variety of different aqueous two-phase systems, e.g. formed by different types of polymers, such as Dextran and PEG or Dextran and Ficoll, or by the same types of polymers with different molecular weights, such as Dextran-70 and PEG-600 or Dextran-70 and PEG-8,000, or by the same polymers but containing different in type and/or concentration salt additives, different buffers of different pH and concentration. The overall partition coefficients for the mixtures determined using particular assay procedure are determined in all the systems and compared. Systems displaying different partition coefficients for the mixtures under comparison may be selected as a separation medium for further fractionation of the mixtures.

The systems displaying different partition coefficients for the mixtures under comparison may be used for fractionation of the mixtures by single-step extraction, multiple-step extraction, column or countercurrent liquid-liquid partition chromatography or other similar procedures. The fractions collected in the separation procedure are analyzed either by partitioning in a single aqueous two-phase system or multiple aqueous two-phase systems or by other means, such as 1-D or 2-D gel electrophoresis, chromatography, or 2-D HPLC with further identification of the structures of the proteins of interest by mass spectrometry or other means.

Generally, the present invention includes one or more of the following steps:

1. Preparation of an aqueous multi-phase system which preferentially and substantially segregates one or more of selected proteins into a single phase, such that it is substantially depleted from the other phases in the system.

2. Preparation of an aqueous multi-phase system such that its different phases differ in their physico-chemical properties to cause a mixture of biomolecules which is put into such a system to partition in accordance to the interaction of each of its constituents or their complexes with each of the phases.

3. Preparation of an aqueous multi-phase system which simultaneously provides properties of both the first and second examples above.

4. Adding a sample containing a mixture of biomolecules into such systems and causing the systems to partition the constituents of such mixtures.

5. Taking aliquots from one or more of the phases of the systems and analyzing the constituents according to standard proteomics techniques knows to those skilled in the art.

The quantity of the biomolecules that is used for each experiment can be greater than 1, 2, 3, 5, 10, 15, 20, 30, 50, 100, 250, 400, 600 and 800 pico-, nano- or micrograms. The quantity of the biomolecules that is used for each experiment can be less than 2, 3, 5, 10, 15, 20, 30, 50, 100, 250, 400, 600, 800 and 1000 micro-, nano- or pico-grams. The volume of the experiment can be greater than 1, 2, 3, 5, 10, 15, 20, 30, 50, 100, 250, 400, 600 and 800 pico-, nano-, micro- or milliliters. The volume of each experiment can be less than 2, 3, 5, 10, 15, 20, 30, 50, 100, 250, 400, 600, 800 or 1000 pico-, nano-, micro-, or milliliters.

It will be appreciated by those skilled in the art that the particular volumes and amounts of protein or solution ingredients employed will vary without limitation according to the biomolecules, its concentrations, and the desired experimental protocol.

### EXPERIMENTAL EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1

In this example it was demonstrated that aqueous two-phase partitioning may be used for segregation of human serum albumin from human serum into one of the phases, while maintaining the albumin and its complexes with ligands available for further analysis.

Human serum samples were obtained from Sigma Chemical Company (St. Louis, Mo., USA) and used without further purification. Poly(ethylene glycol) with molecular weight 600, o-phthaldialdehyde reagent (complete), sodium sulfate, sodium thiocyanate, sodium phosphate, and potassium phosphate were purchased from Sigma Chemical Company (St. Louis, Mo., USA) and used without further purification.

The aqueous two-phase system contained 15.7 wt. % PEG-600 (molecular weight of about 600), 9.5 wt. % sodium sulfate, 2.9 wt. % NaSCN, and 1.2 wt. % sodium/potassium phosphate buffer (pH 7.4). Each system was prepared by mixing the appropriate amounts of stock polymer and buffer solutions dispensed by liquid handling workstation Hamilton ML-4000 into a 5 mL tube. A total volume of 750 microliters was dispensed to the tube, and 250 microliters of serum (without dilution) was added to a system. The ratio between the volumes of the two phases of each system of a final volume of 1.00 mL was as 1:1. The system was shaken vigorously and then centrifuged for 30 min. at about 1700 rpm to speed resolution of the two phases. Tubes were then taken from the centrifuge, and aliquots of 300 microliters volume from the top and the bottom phases were withdrawn in duplicate. Each aliquot from the upper phase was diluted 5-fold with water, and each aliquot from the bottom phase was diluted 10-fold with water, mixed, and used for the SDS-PAGE analysis as described below.

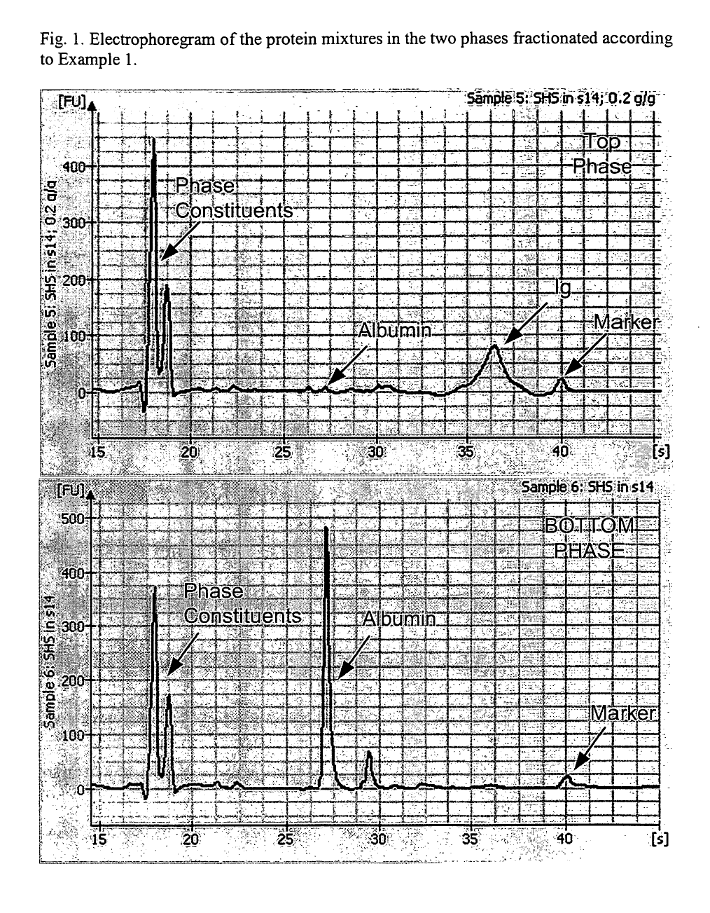

SDS-PAGE procedure was performed on a microchip using Bioanalyzer 2100 (Agilent Technologies) and reagents and microchips from Protein 200 plus assay (Agilent Technologies) using non-reducing conditions according to the standard protocol supplied by the manufacturer. The results obtained are presented in FIG. 1. These results, when integrated by the analysis software of the instrument, indicate that 99.7% of all human serum albumin is concentrated in the bottom phase, while the upper phase contains 0.3% of albumin from initial serum sample.

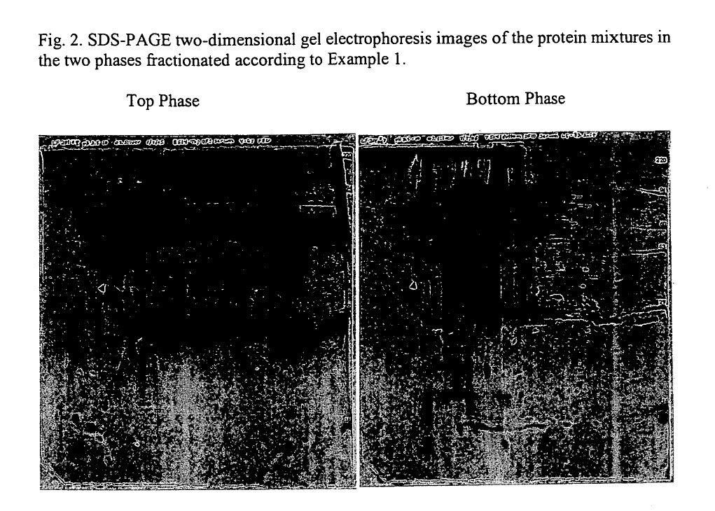

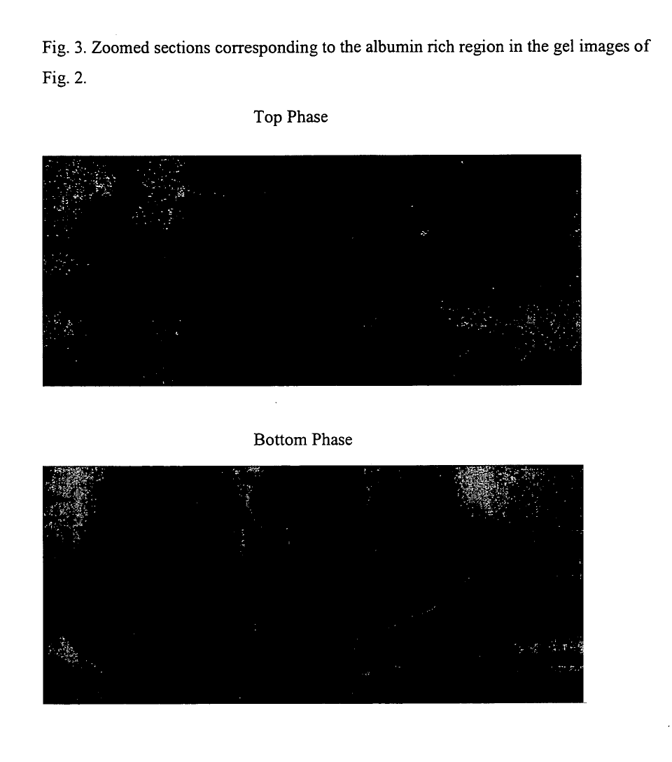

These results were qualitatively confirmed by a 2D-gel electrophoresis analysis performed by Kendrick Laboratories (Madison, Wis.) using aliquots from the top and bottom phases diluted as indicated above. The diluted aliquot from the bottom phase was microdialyzed overnight at 4° C. using the 8,000 molecular weight cutoff membrane. Aliquots from the top and bottom phases were lyophilized and redissolved in SDS Boiling Buffer containing 5% sodium dodecyl sulfate (SDS), 5% beta-mercaptoethanol, 10% glycerol, and 60 mM Tris, pH 6.8, to an overall protein concentration of 3 mg/ml, and heated in a boiling water bath for 5 min before loading. 100 microliters of each solution was loaded and 2D gel electrophoresis was performed using standard isoelectric focusing tube gels containing 2% pH 4-8 BDH ampholines as first dimension, and large format (20×22 cm) 2-D gel. Standard silver staining procedure with preliminary glutaraldehyde treatment of the slab gel to fix proteins by cross linking was performed. Molecular weight standards (220,000, 94,000, 60,000, 43,000, 29,000, and 14,000) were used. The gels were air dried between cellophane sheets. The Afga Arcus II scanner was used to obtain the gel images of the protein fractions in the top and bottom phases, presented in FIGS. 2 and 3, the latter representing a zoomed region corresponding to the albumin fraction in both phases. The images clearly indicate that the albumin depletion results in unmasking a variety of proteins in the albumin-poor serum fraction in the top phase (zoom-in zone).

### Example 2

In this example it was demonstrated that aqueous two-phase partitioning may be used for segregation of animal serum albumin from animal serum into one of the phases, while maintaining the albumin and its complexes with ligands available for further analysis.

Rabbit serum samples were obtained from Sigma Chemical Company (St. Louis, Mo., USA) and used without further purification. Poly(ethylene glycol) with molecular weight 600, o-phthaldialdehyde reagent (complete), sodium sulfate, sodium thiocyanate, sodium phosphate, and potassium phosphate were purchased from Sigma Chemical Company (St. Louis, Mo., USA) and used without further purification.

The aqueous two-phase system contained 15.7 wt. % PEG-600 (molecular weight of about 600), 9.5 wt. % sodium sulfate, 2.9 wt. % NaSCN, and 1.2 wt. % sodium/potassium phosphate buffer (pH 7.4). Each system was prepared by mixing the appropriate amounts of stock polymer and buffer solutions dispensed by liquid handling workstation Hamilton ML-4000 into a 5 mL tube. A total volume of 750 microliters was dispensed to the tube, and 250 microliters of serum (without dilution) was added to a system. The ratio between the volumes of the two phases of each system of a final volume of 1.00 mL was as 1:1. The system was shaken vigorously and then centrifuged for 30 min. at about 1700 rpm to speed resolution of the two phases. Tubes were then taken from the centrifuge, and aliquots of 300 microliters volume from the top and the bottom phases were withdrawn in duplicate. Each aliquot from the upper phase was diluted 5-fold with water, and each aliquot from the bottom phase was diluted 10-fold with water, mixed, and used for the SDS-PAGE analysis as described below.

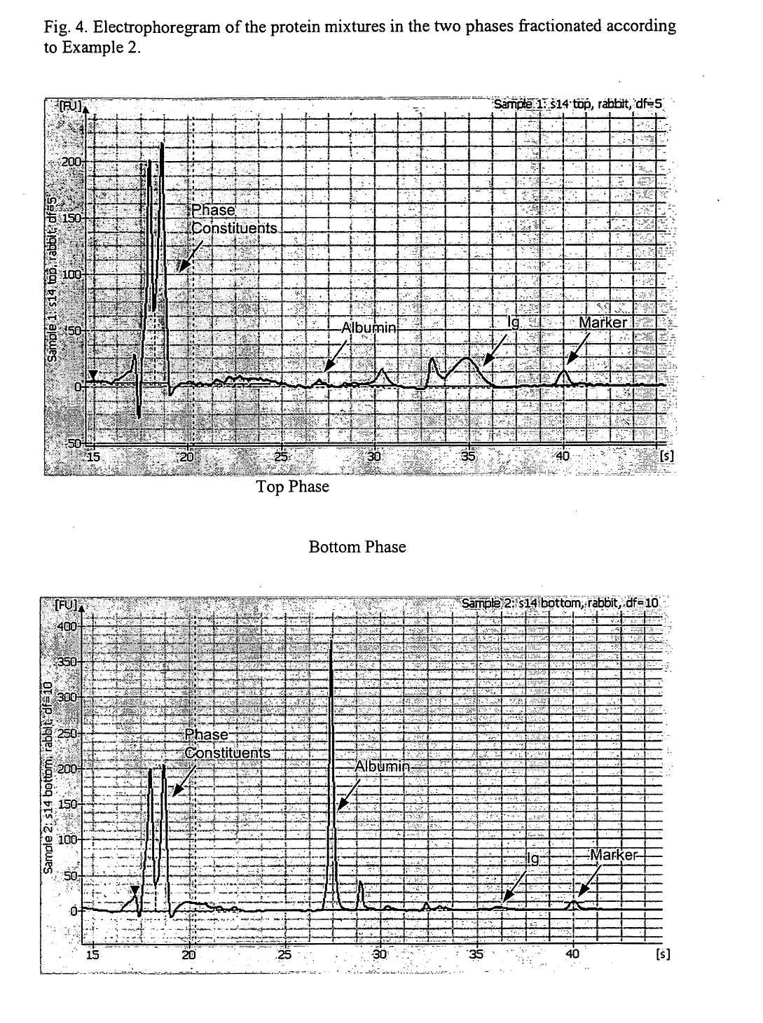

SDS-PAGE procedure was performed on a microchip using Bioanalyzer 2100 (Agilent Technologies) and reagents and microchips from Protein 200 plus assay (Agilent Technologies) using non-reducing conditions according to the standard protocol supplied by the manufacturer. The results obtained are presented in FIG. 4. These results indicate that 99.0% of all rabbit serum albumin is concentrated in the bottom phase, while the upper phase contains 1.0% of albumin from initial serum sample.

### Example 3

In this example it was demonstrated that aqueous two-phase partitioning may be used for depletion of immunoglobulins (IgG) from human serum simultaneously with fractionating the remaining proteins of serum into two fractions available for further analysis.

Human serum samples were obtained from Sigma Chemical Company (St. Louis, Mo., USA) and used without further purification. Poly(ethylene glycol) with molecular weight 600, poly(ethylene glycol) with molecular weight 1450, o-phthaldialdehyde reagent (complete), sodium phosphate, and potassium phosphate were purchased from Sigma Chemical Company (St. Louis, Mo., USA) and used without further purification.

The aqueous two-phase system contained 3.0 wt. % PEG-600 (molecular weight of about 600), 14.0 wt. % PEG-1450 (molecular weight of about 1450), and 15.2 wt. % sodium/potassium phosphate buffer (pH 7.4). Each system was prepared by mixing the appropriate amounts of stock polymer and buffer solutions dispensed by liquid handling workstation Hamilton ML-4000 into a 5 mL tube. A total volume of 750 microliters was dispensed to the tube, and 450 microliters of serum (without dilution) was added to a system. The ratio between the volumes of the two phases of each system of a final volume of 1.00 mL was as 1:1. The system was shaken vigorously and then centrifuged for 30 min. at about 1700 rpm to speed resolution of the two phases. Tubes were then taken from the centrifuge, and aliquots of 300 microliters volume from the top and the bottom phases were withdrawn in duplicate. Each aliquot from the upper phase was diluted 5-fold with water, and each aliquot from the bottom phase was diluted 10-fold with water, mixed, and used for the SDS-PAGE analysis as described below. There was a precipitate of IgG formed at the interface, and this precipitate could be collected and redissolved for further fractionation and/or analysis.

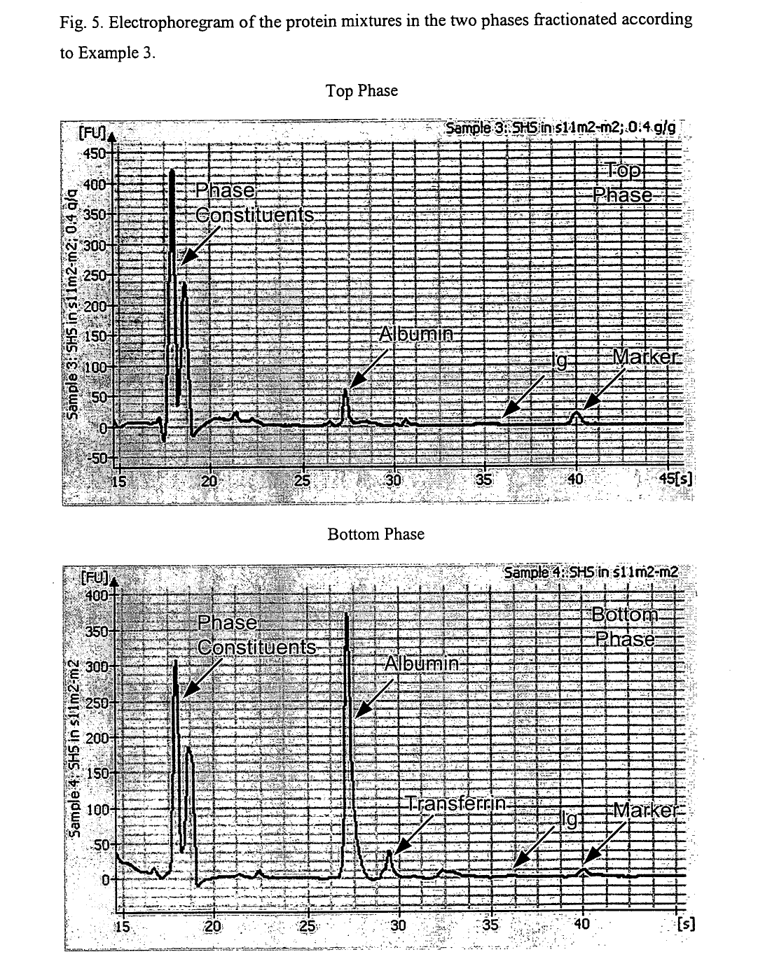

SDS-PAGE procedure was performed on a microchip using Bioanalyzer 2100 (Agilent Technologies) and reagents and microchips from Protein 200 plus assay (Agilent Technologies) using non-reducing conditions according to the standard protocol supplied by the manufacturer. The results obtained are presented in FIG. 5. These results indicate that there is essentially no IgG either in the bottom phase, or in the upper phase.

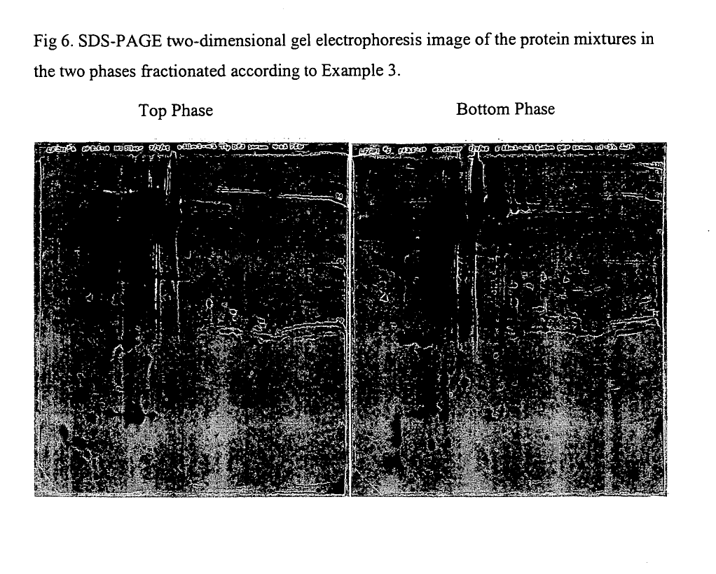

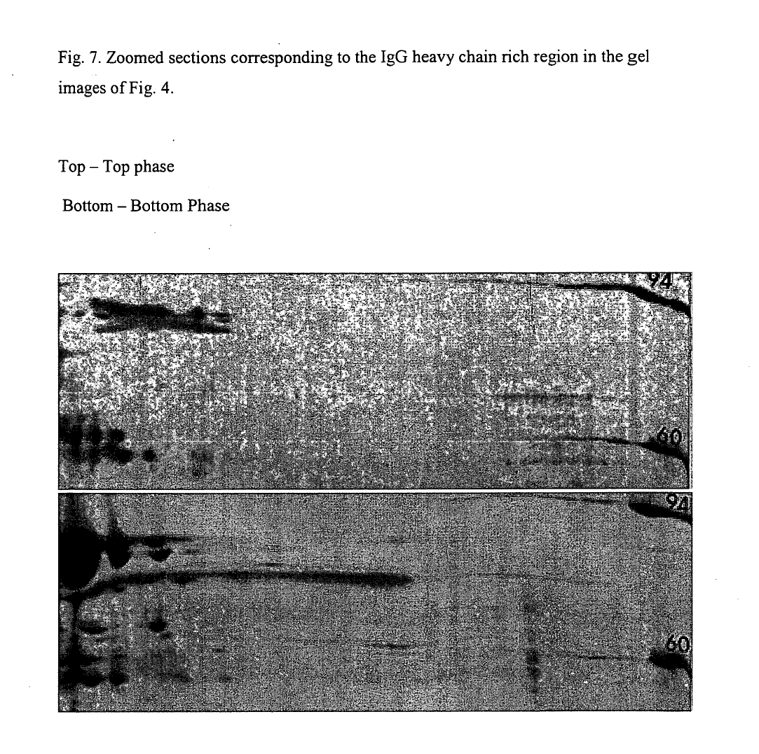

These results were confirmed by 2D-gel electrophoresis analysis performed in Kendrick Laboratories (Madison, Wis.) using aliquots from the top and bottom phases diluted as indicated above. The diluted aliquot from the bottom phase was microdialyzed overnight at 4° C. using the 8,000 molecular weight cutoff membrane. After that the aliquot from the bottom phase and the aliquot from the top phase were lyophilized and redissolved in SDS Boiling Buffer containing 5% sodium dodecyl sulfate (SDS), 5% beta-mercaptoethanol, 10% glycerol, and 60 mM Tris, pH 6.8, to the overall protein concentration of 3 mg/ml, and heated in a boiling water bath for 5 min before loading. 100 microliters of each solution was loaded and 2D gel electrophoresis was performed using standard isoelectric focusing tube gels containing 2% pH 4-8 BDH ampholines as first dimension, and large format (20×22 cm) 2-D gel. Standard silver staining procedure with preliminary glutaraldehyde treatment of the slab gel to fix proteins by cross linking was performed. Molecular weight standards (220,000, 94,000, 60,000, 43,000, 29,000, and 14,000) were used. The gels were air dried between cellophane sheets. The Afga Arcus II scanner was used to obtain the gel images of the protein fractions in the top and bottom phases, presented in FIGS. 6 and 7, the latter representing a zoomed region corresponding to the IgG fraction. The images clearly indicate that there were no spots corresponding to different isoforms of IgG in both serum fraction in both top and bottom phases. The results also illustrate that proteins in the serum were fractionated into the top and bottom phases in accordance with their properties and the properties of the aqueous partitioning system, thus providing further simplification of the total serum before proteomics analysis.

### Example 4

In this example it was demonstrated that aqueous two-phase partitioning may be used for fractionation of human serum, such that both fractions represent subsets of the total sample, and could be used in further proteomics analysis.

Human serum samples were obtained from Sigma Chemical Company (St. Louis, Mo., USA) and used without further purification. Poly(ethylene glycol) with molecular weight 600, o-phthaldialdehyde reagent (complete), sodium chloride, sodium phosphate monobasic, and sodium phosphate dibasic were purchased from Sigma Chemical Company (St. Louis, Mo., USA) and used without further purification. Dextran-70 with molecular weight of ˜70,000 was obtained from USB Corp. (Cleveland, Ohio, USA) and used without further purification.

The aqueous two-phase system contained 15.8 wt. % PEG-600 (molecular weight of about 600), 11.9 wt. % dextran-70 (molecular weight of about 70,000), 0.15 M NaCl, and 0.01 M sodium phosphate buffer (pH 7.4). Each system was prepared by mixing the appropriate amounts of stock polymer and buffer solutions dispensed by liquid handling workstation Hamilton ML-4000 into a 5 mL tube. A total volume of 550 microliters was dispensed to the tube, and 450 microliters of serum (without dilution) was added to a system. The ratio between the volumes of the two phases of each system of a final volume of 1.00 mL was as 1:1. The system was shaken vigorously and then centrifuged for 30 min. at about 3400 rpm to speed resolution of the two phases. Tubes were then taken from the centrifuge, and aliquots of 300 microliters volume from the top and the bottom phases were withdrawn in duplicate. Each aliquot from the upper phase was diluted 5-fold with water, and each aliquot from the bottom phase was diluted 10-fold with water, mixed, and used for the SDS-PAGE analysis as described below.

SDS-PAGE procedure was performed on a microchip using Bioanalyzer 2100 (Agilent Technologies) and reagents and microchips from Protein 200 plus assay (Agilent Technologies) using non-reducing conditions according to the standard protocol supplied by the manufacturer. Results indicate different protein composition in the bottom phase and in the upper phase (not depicted in the Figures).

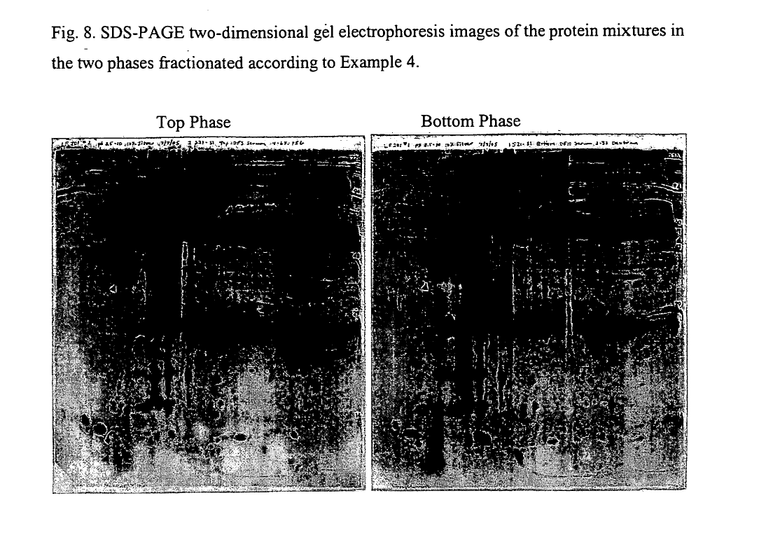

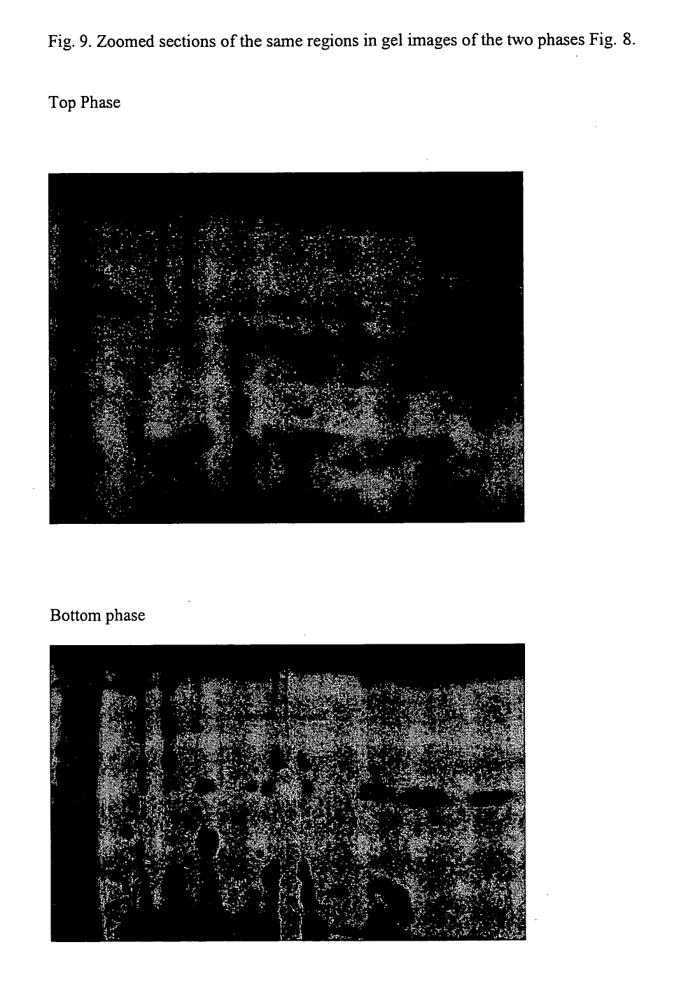

These results were confirmed by 2D-gel electrophoresis analysis performed in Kendrick Laboratories (Madison, Wis.) using aliquots from the top and bottom phases diluted as indicated above. The diluted aliquot from the bottom phase was microdialyzed overnight at 4° C. using the 8,000 molecular weight cutoff membrane. After that the aliuot from the bottom phase and the aliquot from the top phase were lyophilized and redissolved in SDS Boiling Buffer containing 5% sodium dodecyl sulfate (SDS), 5% beta-mercaptoethanol, 10% glycerol, and 60 mM Tris, pH 6.8, to the overall protein concentration of 3 mg/ml, and heated in a boiling water bath for 5 min before loading. 100 microliters of each solution was loaded and 2D gel electrophoresis was performed using standard isoelectric focusing tube gels containing 2% pH 4-8 BDH ampholines as first dimension, and large format (20×22 cm) 2-D gel. Standard silver staining procedure with preliminary glutaraldehyde treatment of the slab gel to fix proteins by cross linking was performed. Molecular weight standards (220,000, 94,000, 60,000, 43,000, 29,000, and 14,000) were used. The gels were air dried between cellophane sheets. The Afga Arcus II scanner was used to obtain the gel images of the protein fractions in the top and bottom phases, presented in FIGS. 8 and 9, the latter representing a zoomed section of the image in the two phases. The images clearly indicate that the different subsets of proteins are concentrated in the bottom and upper phases, thus providing simplification of the total serum proteome before proteomics analysis. The basis for the present fractionation prior to the size and charge gel fractionation is thus different from size and charge alone.

### Example 5

In this example it was demonstrated that sequential aqueous two-phase partitioning may be used for depletion of animal serum of immunoglobulins and transferrin first from a single phase of a first partitioning system, followed by a second partitioning to remove albumin, thus resulting in a single phase of the second system that is depleted of both albumin, globulins, and transferrin and while maintaining the depleted albumin and its complexes, globulins and transferrin available for further analysis. This example further demonstrates that this procedure with the same aqueous two-phase partitioning system compositions is equally efficient at removing albumin, globulins, and transferrin from sera obtained from different species.

Sera samples from bovine, rat, and rabbit were obtained from Lampire Biological Laboratories (Piperseville, Pa., USA) and used without further purification. Poly(ethylene glycol) with molecular weight 600, poly(ethylene glycol) with molecular weight 1450, o-phthaldialdehyde reagent (complete), sodium sulfate, sodium thiocyanate, sodium phosphate, and potassium phosphate were purchased from Sigma Chemical Company (St. Louis, Mo., USA) and used without further purification.

The first aqueous two-phase system contained 15.7 wt. % PEG-600 (molecular weight of about 600), 9.5 wt. % sodium sulfate, 2.9 wt. % NaSCN, and 1.2 wt. % sodium/potassium phosphate buffer (pH 7.4). Each system was prepared by mixing the appropriate amounts of stock polymer and buffer solutions dispensed by liquid handling workstation Hamilton ML-4000 into a 5 mL tube. A total volume of 750 microliters was dispensed to the tube, and 250 microliters of serum (without dilution) was added to a system. The ratio between the volumes of the two phases of each system of a final volume of 1.00 mL was as 1:1. The system was shaken vigorously and then centrifuged for 15 min. at about 1700 rpm to speed resolution of the two phases. Tubes were then taken from the centrifuge, and aliquots of 200 microliters volume from the top and the bottom phases were withdrawn in duplicate.

The aliquot of 180 microliter volume from the top phase was added to the second two-phase system. The second aqueous two-phase system contained 3.0 wt. % PEG-600 (molecular weight of about 600), 14.0 wt. % PEG-1450 (molecular weight of about 1450), and 15.2 wt. % sodium/potassium phosphate buffer (pH 7.4). Each system was prepared by mixing the appropriate amounts of stock polymer and buffer solutions dispensed by liquid handling workstation Hamilton ML-4000 into a 5 mL tube. A total volume of 500 microliters was dispensed to the tube, and 180 microliters of the aliquot from the top phase of the first two-phase system was added to the second system. The ratio between the volumes of the two phases of each system of a final volume of 0.68 mL was as 1:1. The system was shaken vigorously and then centrifuged for 15 min. at about 1700 rpm to speed resolution of the two phases. Tubes were then taken from the centrifuge, and aliquots of 200 microliters volume from the top and the bottom phases were withdrawn in duplicate. Each aliquot from the upper phase was diluted 5-fold with water, and each aliquot from the bottom phase was diluted 10-fold with water, mixed, and used for the SDS-PAGE analysis as described below. There was a precipitate of IgG formed at the interface, and this precipitate could be collected and redissolved for further fractionation and/or analysis.

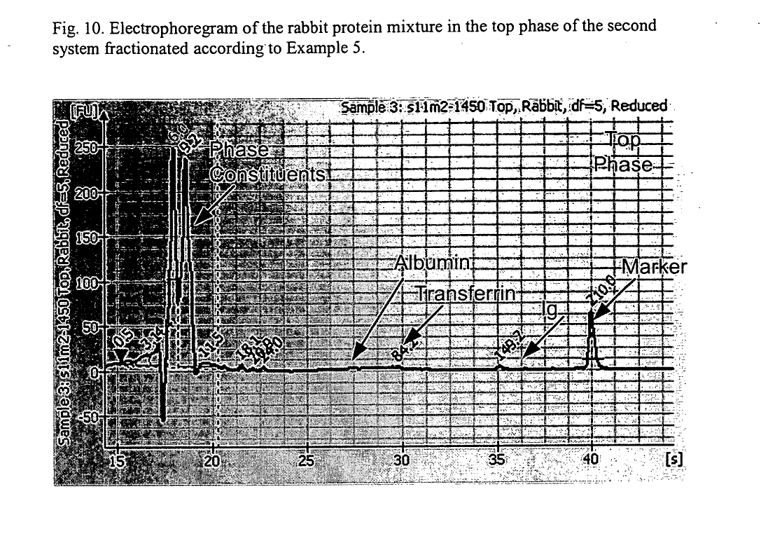

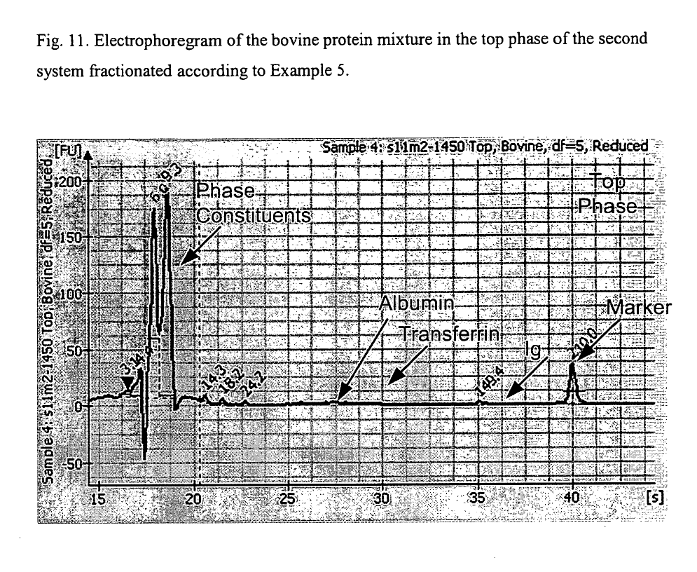

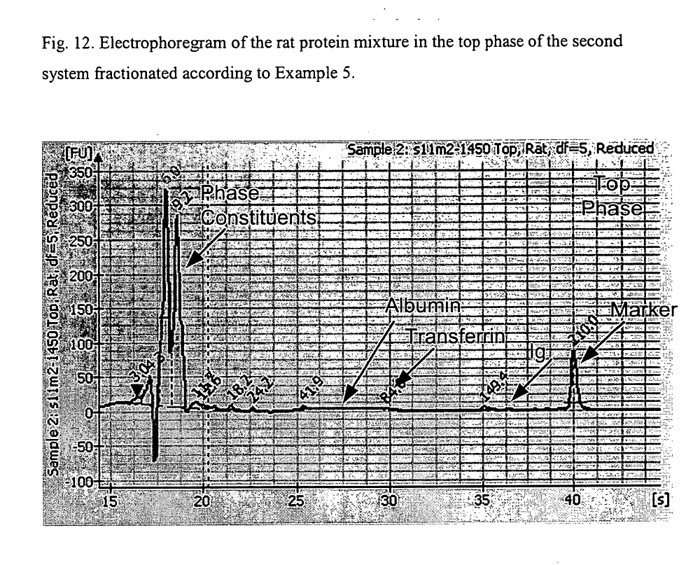

SDS-PAGE procedure was performed on a microchip using Bioanalyzer 2100 (Agilent Technologies) and reagents and microchips from Protein 200 plus assay (Agilent Technologies) using non-reducing conditions according to the standard protocol supplied by the manufacturer. The results obtained are presented in FIGS. 10-12. These results indicate that no albumin, globulins, or transferrin could be detected in the upper phase of the second system, for each of the three animal species provided.

Using the procedures of the present invention, using either a single fractionation or multiple or sequential fractionations, preferably the concentration of albumin, haptoglobin, immunoglobulins, transferrin, lipoprotein and/or the other abundant proteins mentioned herein is reduced at least: 60, more preferably 70, more preferably 80, more preferably 90, more preferably 95, more preferably 98, more preferably 99, more preferably 99.8, more preferably 99.9, percent. For example, the concentrations of albumin, immunoglobulins, and/or transferrin can be reduced at least 95% using different aqueous partitioning systems; for example, with regard to sera obtained from different species such as human, rat, rabbit, and bovine, without changing of the system composition.

Multiple and sequential fractionation procedures according to the invention can be performed to provide refinement and purification. For example, a first fractionation procedure can be performed, resulting in Fraction A. Fraction A can then be fractionated, resulting in Fraction B. Fraction B can then be fractionated, resulting in Fraction C. This can be continued sequentially, resulting in Fractions D, E, F, etc. For example, 2, 3, 4, 5, 6, 7, etc, sequential fractionation procedures can be performed to provide enhanced purification.

The present invention provides several advantages for proteomics analysis as follows:

1. Depletion of specific abundant proteins from a mixture.

2. Depletion of specific abundant proteins from a mixture, while providing for simultaneous access of both the depleted protein(s) and the mixture for further analysis.

3. Providing a fractionation dimension of a mixture based on a physic-chemical property that is not based solely on charge or molecular weight.

4. Providing a sequential fractionation capability.

5. Providing a fractionation method whereas the entire sample is available for further analysis between the two or more fractions.

6. Providing for liquid phase fractionation that maintains protein structure, in particular, does not denature proteins, and potentially maintains protein-protein and protein-ligand interactions as a basis for fractionation.

What is claimed is:

1. A method for proteomics analysis, including fractionation of a mixture of biomolecules, said mixture containing at least a first biomolecule and a second biomolecule, said method comprising the steps of:
providing a multi-phase partitioning system;
combining a sample containing said mixture of biomolecules with said system;
causing or permitting said system to separate into at least a first phase and a second phase, wherein said first biomolecule is preferentially segregated into said first phase;
selecting said first phase or said second phase; and
performing further proteomics analysis on said selected phase.

2. The method of claim 1, wherein the partitioning system is an aqueous multi-phase partitioning system.

3. The method of claim 1, wherein the partitioning system is an aqueous two-phase partitioning system.

4. The method of claim 1, wherein at least one of the components of the partitioning system is a polymer.

5. The method of claim 1, wherein at least one of the components of the partitioning system is a salt.

6. The method of claim 1, wherein at least one of the components of the partitioning system is a surfactant.

7. The method of claim 1, wherein the pH of said sample under analysis is within the range of pH from 2.0 to 10.0.

8. The method of claim 1, wherein the temperature of the system during separation is in the range of 4° C. to 60° C.

9. The method of claim 1, wherein the partitioning system is an aqueous partitioning polymer/polymer system comprising dextran and polyethylene glycol.

10. The method of claim 1, wherein the partitioning system is an aqueous partitioning polymer/polymer system comprising dextran and polyvinylpyrrolidone.

11. The method of claim 1, wherein the partitioning system is an aqueous partitioning polymer/salt system comprising polyethylene glycol and salt.

12. The method of claim 1, wherein the partitioning system is an aqueous partitioning polymer/salt system comprising polyethylene glycol and buffer, said buffer comprising phosphate, citrate, borate, and/or other ions.

13. The method of claim 1, wherein the partitioning system is an aqueous partitioning polymer/salt system comprising polyvinylpyrrolidone and salt.

14. The method of claim 1, wherein the partitioning system is an aqueous partitioning polymer/salt system comprising polyvinylpyrrolidone and buffer, said buffer comprising phosphate, citrate, borate, and/or other ions.

15. The method of claim 1, wherein the concentration of said first biomolecule is reduced at least 60 percent from said sample to said second phase.

16. The method of claim 1, wherein the concentration of said first biomolecule is reduced at least 95 percent from said sample to said second phase.

17. The method of claim 1, wherein said first biomolecule is selected from the group consisting of albumin, haptoglobin, immunoglobulins, transferrin and lipoprotein.

18. The method of claim 1, further comprising a step of performing a fractionation procedure on said selected phase to yield a second selected phase.

19. The method according to claim 18, further comprising a step of performing a fractionation procedure on said second selected phase to yield a third selected phase.

20. The method of claim 1, wherein said multi-phase partitioning system separates said mixture of biomolecules based upon relative hydrophobicity of said biomolecules.

21. The method of claim 1, wherein substantially all of the biomolecules in the mixture of biomolecules are separated into the different phases of said multi-phase system and are available for further proteomics analysis.

22. The method of claim 1, wherein the fractionation substantially preserves pre-existing non-covalent interactions between biomolecules in the mixture or between biomolecules and ligands in the mixture.
